(19) United States
(12) Patent Application Publication
(10) Pub. No.: US 2014/0057884 A1
(43) Pub. Date: Feb. 27, 2014

(54) PHARMACEUTICAL COMPOSITION FOR PREVENTING OR TREATING LIVER CANCER COMPRISING STIGMASTEROL AND 5 BETA-HYDROXYSITOSTANOL ISOLATED FROM NAVICULA INCERTA

(71) Applicant: PUKYONG NATIONAL UNIVERSITY INDUSTRY-UNIVERSITY CO, Busan (KR)

(72) Inventors: Se-Kwon KIM, Busan (KR); Kyong-Hwa KANG, Busan (KR); Young-Sang KIM, Busan (KR)

(73) Assignee: Pukyong National University Industry-University Cooperation Foundation, Busan (KR)

(21) Appl. No.: 13/665,575

(22) Filed: Oct. 31, 2012

(30) Foreign Application Priority Data
Aug. 23, 2012 (KR) 10-2012-0092353

(51) Int. Cl. A61K 31/575

(52) U.S. Cl. CPC A61K 31/575
USPC 514/182

(57) ABSTRACT

A novel use of phytosterol isolated from Navicula incerta having effect of preventing or treating liver cancer is provided. Accordingly, a composition for preventing or treating liver cancer including stigmasterol and 5β-hydroxysitostanol isolated from Navicula incerta as an active ingredient is provided. Accordingly, and 5β-hydroxysitostano upregulate expression of genes involved with hepatoma cell apoptosis inducing factors, while downregulating genes involved with hepatoma cell apoptosis inhibiting genes, thereby lead into effective prevention or treatment of liver cancer.

### CROSS-REFERENCES TO RELATED APPLICATION

This application claims priority from Korean Patent Application No. 10-2012-0092353, filed on Aug. 23, 2012, in the Korean Intellectual Property Office, the contents of which are incorporated herein by reference in its entirety

### BACKGROUND OF THE INVENTION

1. Field of the Invention

The present invention relates a novel use of stegmasterol and 5 beta-hydroxysitostanol isolated from Navicul incerta which present effect of preventing or treating liver cancer.

2. Description of the Related Art

Liver cancer is one of the most lethal cancers around the world, but it is particularly the very serious concern in Asian countries including South Korea. Liver cancer is the fifth most frequent diseases around the world, marking the third largest population who die from this. The liver cancer is particularly dangerous because unapparent symptoms of this disease in many times deceive patients from early detection thereof, which in turn cause low treatment and survival rate.

Numerous attempts have been made to treat cancers, which can be mainly categorized into chemotherapy, surgery, and radiation therapy. Chemotherapy mainly involves oral or intravenous administration of anticancer agent, but because the drug is circulated throughout the patient's body in bloodstreams before finally reaching the lesion. This means that only a minute amount of the drug is accumulated at the lesion. Accordingly, more drugs are administered to ensure that the effective amount of drug reaches the lesion, but this accompanies severe side effects. Further, anticancer agents with relatively better effects are accompanied with severer side effect such as pains or fever. Radiation therapy is particularly accompanied with the risk of damaging even normal cells near the cancer cells, which can bring in complications. Therefore, the therapy is problematic in view of safety.

Given this, the world including Asia has increasing attention for the alternative medicine based on natural product which is generally safer and has less concerns of side effects. The anticancer substance is particularly originated from the natural product or from the synthetic derivative isolated mainly from natural product. However, despite the continuous efforts to develop a cure for the liver cancer from the natural product, a few substances are currently used for the treatment or in the clinical trial.

Accordingly, the present inventors have discovered stigmasterol and 5β-hydroxysitostanol isolated from Navicul incerta ultimately enable treatment of liver cancer by upregulating the genes involved with the apoptosis-inducing factor and downregulating the genes involved with the apoptosis inhibitive factor.

### SUMMARY OF THE INVENTION

An object of the present invention is to provide a method of preventing and treating liver cancer comprising administering to a subject in need thereof a stigmasterol or 5β-hydroxysitostanol as an active ingredient It is another object of the present invention is to provide a composition for the prevention and treatment of liver cancer, comprising stegmasterol and/or 5β-hydroxysitostanol isolated from Navicula incerta as an active ingredient.

In order to achieve the above-mentioned object, the present invention provides a method of preventing and treating liver cancer comprising administering to a subject in need thereof a stigmasterol or 5β-hydroxysitostanol as an active ingredient

To achieve the above objects, there is provided a pharmaceutical composition for the prevention and treatment of liver cancer, which includes stigmasterol isolated from Navicula incerta as an active component.

In order to achieve the above-mentioned object, the present invention provides a pharmaceutical composition for the prevention and treatment of liver cancer, which includes 5 beta-hydroxysitostanol isolated from Navicula incerta as an active component.

In one embodiment, the stigmasterol and 5β-hydroxysitostanol may upregulate expression of a gene selected from a group consisting of caspase-8, 9, Bax, p53, p21, FAS and FASL of human hepatoma cells.

In one embodiment, the stigmasterol and 5β-hydroxysitostanol may downregulate expression of a gene selected from a group consisting of Bcl-2 and XIAP of human hepatoma cells.

According to embodiments, stigmasterol and 5β-hydroxysitostanol isolated from Navicula incerta may upregulate expression of caspase-8, 9, Bax, p53, p21, FAS and FASL which are apoptosis inducing factors of hepatoma cells, and downregulate expression of Bcl-2 and XIAP which are apoptosis inhibitory factors, to thereby effectively prevent or treat liver cancer.

Further, the present invention can be efficaciously used as a treatment that can prevent or treat liver cancer, particularly because the synthetic inducer isolated from the natural product provides excellent biostability and accordingly does not induce various side effects from toxicity which are usually generated by the conventional liver cancer treatments.

### BRIEF DESCRIPTION OF THE DRAWINGS

The above and/or other aspects and advantages of the present invention will become apparent and more readily appreciated from the following detailed description, taken in conjunction with the accompanying drawings of which:

FIG. 1 is a graph showing cell viability of HepG2 cells treated with stigmasterol and 5β-hydroxysitostanol for 24 h according to the present invention, in which different letters (a-c) on each bar shows significant difference (p<0.05) by Duncan's multiple range test;

FIG. 2 shows morphological changes of stigmasterol treated HepG2 cells and 5β-hydroxysitostanol treated HepG2 cells according to the present invention, in which (a) shows the morphological changes detected under light microscope (viewed at magnification of 100×) and (b) shows the morphological changes detected under fluorescence microscope (viewed at magnification of 400×) after Hoechst 33342 staining;

FIGS. 3a and 3b are graphs illustrating result of cycle analysis of hepatoma cells treated with stigmasterol and 5β-hydroxysitostanol according to the present invention, respectively;

FIGS. 4a and 4b show result of RT-PCR analysis on stigmasterol and 5β-hydroxysitostanol treated hepatoma cell gene according to the present invention, in which β-actin is used as a control;

FIG. 5 shows result of Western blot analysis on hepatoma cells treated with stigmasterol and 5β-hydroxysitostanol according to the present invention, in which β-tubulin is used as a control.

### DETAILED DESCRIPTION OF THE INVENTION

Reference will now be made in detail to the embodiments of the present invention, examples of which are illustrated in the accompanying drawings, wherein like reference numerals refer to the like elements throughout. The embodiments are described below to explain the present invention by referring to the figures.

An embodiment of the present invention provides a composition for the prevention and treatment of liver cancer, comprising stigmasterol and/or 5β-hydroxysitostanol isolated from Navicula incerta as an active ingredient.

Navicula incerta is microalgae. Stigmasterol and 5β-hydroxysitostanol isolated from Navicula incerta belong to phytosterol family, and phytosterol is known for its inhibitory effect of cholesterol in blood vessel cells. Further, phytosterol was also reported for its apoptosis inducing effect in lung cancer, prostate cancer, and colon cancer HT 29 cell and U937 human monocyte. However, little has been discovered about the apoptosis-inducing effect of phytosterol in the cells involved with liver cancer.

Accordingly, the inventors of the present invention investigated and confirmed, for the first time, the liver cancer treatment effect of stigmasterol and 5β-hydroxysitostanol, which are the specific types of phytostreol, by inducing apoptosis of HepG2 cells, the human hepatoma cells.

A method for isolating stigmasterol and 5β-hydroxysitostanol from Navicula incerta according to an embodiment of the present invention will be explained below.

After mass culture, Navicula incerta was lyophilized to powder state. Navicula incerat in powder form was added to Erlenmeyer flask and percolated for 24 h in methanol to give fraction 1 (Fr. 1). Methanol extract was filtered using filter paper, and the remaining powder was percolated using mixed solvent of methanol and dichloromethane for 24 h to give fraction 2 (Fr. 2). The mixed solvent extract was filtered using filter paper, and the remaining powder was percolated for 24 h using dichloromethane and then extracted to give fraction 3 (Fr. 3). Mixed solvent of dichloromethane and acetone was added to Fr. 2 for column chromatography to give 11 fractions (Fr. 2.1-11). Among the fractions, the mixture of Fr. 2.3 and Fr. 2.4 was used for column chromatography using mixed solvent of hexane and ethyl acetate to give whitish solid state stigmaterol. Further, Fr. 2.5 was selected for column chromatography using mixed solvent of hexane and ethyl acetate to give 5β-hydroxysitostanol in whitish solid form.

Stigmasterol isolated from Navicula incerta in the manner explained above is distinguished from cholesterol mainly in view of double bond in the side chain and presence of three cyclohexane rings and one cyclopetnane ring included therein. Stigmasterol has been reported of its strong effect of inhibiting cancer including breast cancer, ovarian cancer, prostate cancer, or colon cancer, and is also known for decreasing cholesterol levels. Structure of stigmasterol is represented by formula 1.

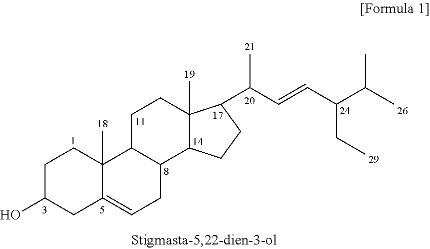 [SMILES: CCC(/C=C/C(C)C1CCC2C3CC=C4CC(O)CCC4(C)C3CCC12C)C(C)C]

Unlike stigmasterol, 5β-hydroxysitostanol isolated from Navicula incerta in the manner explained above does not have double bond, but instead has extra hydroxyl group on C-5 position. The structure of 5β-hydroxysitostanol is represented by formula 2.

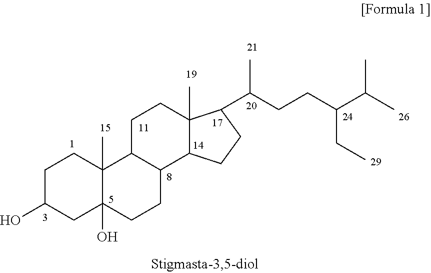 [SMILES: CCC(CCC(C)C1CCC2C3CCC4(O)CC(O)CCC4(C)C3CCC12C)C(C)C]

However, none has been reported about the inducing role of stigmasterol and 5β-hydroxysitostanol on apoptosis of cells related with liver cancer.

Accordingly, the present inventors confirmed liver cancer treatment effect of stigmasterol and 5β-hydroxysitostanol according to the present invention, and this will be explained below.

In one embodiment, viability of human hepatocellular carcinoma (HepG2) cells treated with stigmasterol and 5β-hydroxysitostanol was observed by MTT assay. As a result, it was observed that viability of HepG2 cells decreased as the concentration of the two substances increased (see Experiment Example 1).

According to one embodiment, morphological changes in HepG2 cells treated with stigmasterol and 5β-hydroxysitostanol was observed under microscope. As a result, it was observed that the number of attached HepG2 cells (number of living cells) decreased significantly, and also the shape thereof shrunk. Further, after HepG2 cells staining and observation under fluorescence microscope, it was observed that nucleus of attached HepG2 cells greatly condensed as the concentration of the two substances increased. This is the typical change observed in cell apoptosis (see Experiment Example 2).

In one embodiment, cell cycle of HepG2 cells treated with stigmasterol and 5β-hydroxysitostanol was confirmed by measuring DNA content with FACS. As a result, it was confirmed that G2/M phase of HepG2 cell cycle increased as the concentration of the two substances increased. This indicates the effect of the two substances which inhibit circulation of cell dividing cycle of HepG2 cells, i.e., arrest HepG2 cells at G2/M phase to thereby inhibit proliferation thereof (see Experiment Example 3).

In one embodiment, mRNA expression of HepG2 cells was investigated by RT-PCR and Western blot analysis. As a result, it was confirmed that stigmasterol and 5β-hydroxysitostanol upregulate expression of caspase-8, 9, Bax, p53, p21, FAS and FASL which are cell apoptosis inducing factors, and downregulate expression of Bcl-2 and XIAP which are cell apoptosis inhibiting factors, thereby effectively preventing or treating liver cancer.

Accordingly, stigmasterol and 5β-hydroxysitostanol isolated from Navicula incerta according to the present invention can be efficaciously used in the treatment of liver cancer.

Accordingly, stigmasterol and 5β-hydroxysitostanol isolated from Navicula incerta according to the present invention can be used as a pharmaceutical composition for the prevention or treatment of liver cancer.

The pharmaceutical composition according to the present invention may include a pharmaceutically-effective amount of stigmasterol and/or 5β-hydroxysitostanol singularly, or in combination with one or more pharmaceutically-acceptable carrier, excipient or diluent.

As used herein, the wording “pharmaceutically-effective amount” may refer to a sufficient amount of the bioactive component to be administered into an animal or human to provide intended biological or pharmaceutical activity. However, the “pharmaceutically-effective amount” may vary appropriately depending on age, weight, health condition, gender, route of administration and treatment period of a subject of the administration.

Further, the wording “pharmaceutically-acceptable carrier, excipient or diluent” as used herein may refer to carrier, excipient or diluents which is biologically acceptable and which generally does not cause allergic reaction such as stomach disorder or dizziness or similar reaction.

An example of the carrier, excipient or diluents may include lactose, dextrose, sucrose, sorbitol, manitol, xylitol, erythritol, malthitol, starch, acacia gum, alginate, gelatin, calcium phosphate, calcium silicate, cellulose, methyl cellulose, polyvinyl pyrrolydone, water, methylhydroxybenzoate, propylhydroxybenzoate, talc, magnesium stearate and mineral oil. In addition to the above, the pharmaceutical composition according to the present invention may further include filler, anti-coagulant, lubricant, wetting agent, fragrance, emulsifier, preservative, or the like.

Further, the composition according to the present invention may be formulated into dosage forms according to known method to provide rapid, continuous or delayed release of the active component after administered into a mammalian animal, and the dosage forms may vary for oral or parenteral administration. The dosage forms may include powder, granule, pill, suspension, syrup, aerosol, soft or hard gelatin capsule, sterile solution for injection, or sterile powder.

The representative example of the dosage form for parenteral administration is an injectable dosage form which is preferably an isotonic solution or suspension. The injectable dosage form may be prepared according to known technique using appropriate dispersant or wetting agent and suspending agent. For example, each ingredient may be prepared into a dosage form for injection by dissolving in saline solution or buffer solution. Further, the dosage form for oral administration includes, for example, ingestible tablets, buccal tables, troches, capsules, elixirs, suspensions, syrups, wafers, and/or the like. These dosage forms may include in addition to active ingredient a diluents (e.g., lactose, dextrose, sucrose, manitol, sorbitol, cellulose and/or glycine) and lubricant (e.g., silica, talc, stearate and magnesium or calcium salt thereof and/or polyethylene glycol). The tablet may include a binder such as magnesium aluminum silicate, starch paste, gelatin, tragacanth, methylcellulose, sodium carboxymethylcellulose and/or polyvinyl pyrrolydine, and depending on needs, may additionally include a disintegrating agent such as starch, agar, alginic acid, or sodium salt, absorbent, colorant, flavoring agent and/or sweetening agent. The dosage form may be prepared by general mixing, granulating or coating methods.

Further, the composition according to the present invention may additionally include preservative, water-dispersable powder, emulsion activator, salt for osmotic regulation, or adjuvant such as buffer, or other therapeutically useful materials, and may be prepared into dosage form by known methods.

The examples of the present invention will be explained in detail below with reference to the accompanying drawings. However, the examples are written only for illustrative purpose and may not be construed as limiting of the present invention.

### Example 1

### Isolation of Stigmastreol and 50-Hydroxysitostanol from Navicula incerta

After mass culture, Navicula incerta (strain: KMMCC B-001) was lyophilized to give powder. Navicula incerta in powder form was put into 5 L Erlenmeyer flask, and percolated 24 h in methanol to give fraction 1 (Fr. 1). The methanol extract was filtered using filter paper, and the remaining powder was percolated 24 h in the 3 L mixed solvent of methanol and dichloromethane (1:1) to give fraction 2 (Fr. 2). The mixed solvent extract was filtered using filter paper, and the remaining powder was percolated 24 h in 3 L dichloromethane and extracted to give fraction 3 (Fr. 3).

Using dichloromethane and acetone (30:1 to 1:2), 11 fractions were obtained from fraction 2 (20.313 g) by column chromatography (Fr. 2.1 to 2.11).

Using hexane and ethyl acetate (10:1 to 5:1), 17.2 mg of whitish solid form of stigmasterol was obtained from fractions 2.3 and 2.4 by column chromatography.

Using hexane and ethyl acetate (10:1 to 5:1), 55.8 mg of whitish solid form of 5β-hydroxysitostanol was obtained from fraction 2.5 by column chromatography.

### Example 2

### Cell Culture

Human hepatoma (HepG2) cells were cultured in 10 cm² dish and maintained in DMEM supplemented with 10% fetal bovine serum (FBS). The medium was diluted in 100 μg streptomycin/penicillin per mL. 5% CO₂ was introduced, and cells were subcultured by detaching with trypsin-EDTA solution 1-2 times every week at about 70-80% confluency.

### Experimental Example 1

### Cell Viability Assay

Cell viability was measured using MTT [3-(4,5-dimethylthiazol-2-yl)-2,5-diphenyltetrazolium bromide] assay utilizing colorless MTT is reduced to purple formazan by the action of mitochondrial dehydrogenase which has viability only in living cells.

The HepG2 cells were grown in 96-well plates at a density of 2×10⁴ cells/well. After 24 h, cells were treated with different concentrations (5 μL, 10 μL, 20 μM) of stigmasterol and 5β-hydroxysitostanol. After incubation for 24 h, the cells were incubated with 100 μL of MTT (1 mg/mL) for 4 h. Finally, DMSO was added so that the amount of formazan crystal was determined by measuring the absorbance at 550 nm using a multidetection microplate reader.

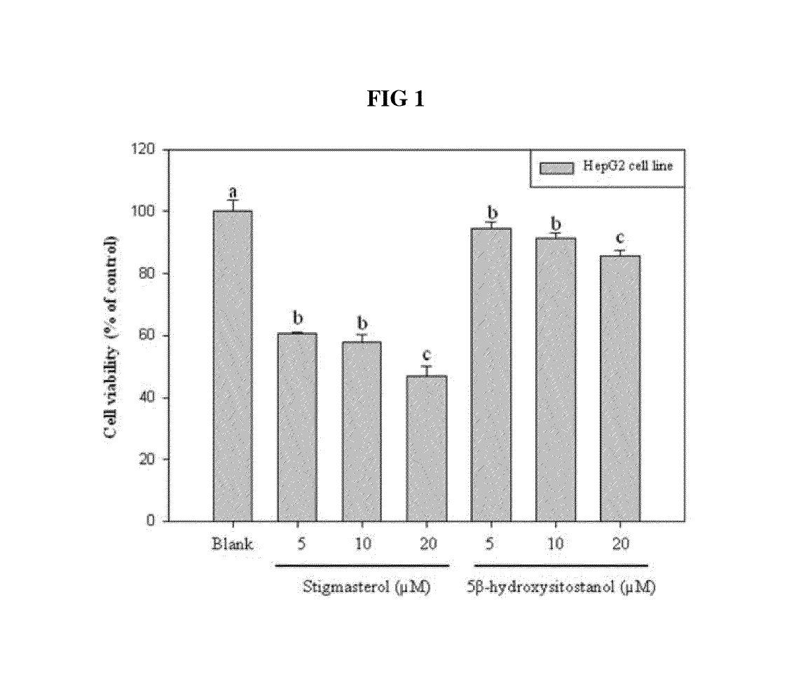

Referring to FIG. 1, exposure of HepG2 cells to increasing concentrations of stigmasterol and 5β-hydroxysitostanol resulted in a dose-dependent decrease in cell viability. Treatment with stigmasterol for 24 h inhibited the cell viability with the rates of approximately 40, 43 and 54% at the concentrations of 5, 10 and 20 μM, respectively. Treatment with 5β-hydroxysitostanol for 24 h inhibited the cell viability with the rates of approximately 6, 9 and 15% at the concentrations of 5, 10 and 20 μM, respectively.

### Experimental Example 2

### Observation of Morphological Changes of the Cells

HepG2 ells were grown in 12-well plates at a density of 2×10⁴ cells/well. After overnight incubation, cells were treated with different concentrations (5 μL, 10 μL, 20 μM) of stigmasterol and 5β-hydroxysitostanol and incubated for another 24 h. The cells were washed with PBS. After washing, 4% (v/v) formaldehyde solution was dissolved in PBS and added to the plate, and left for 1 h at room temperature. The fixed cells were detected by a light microscope.

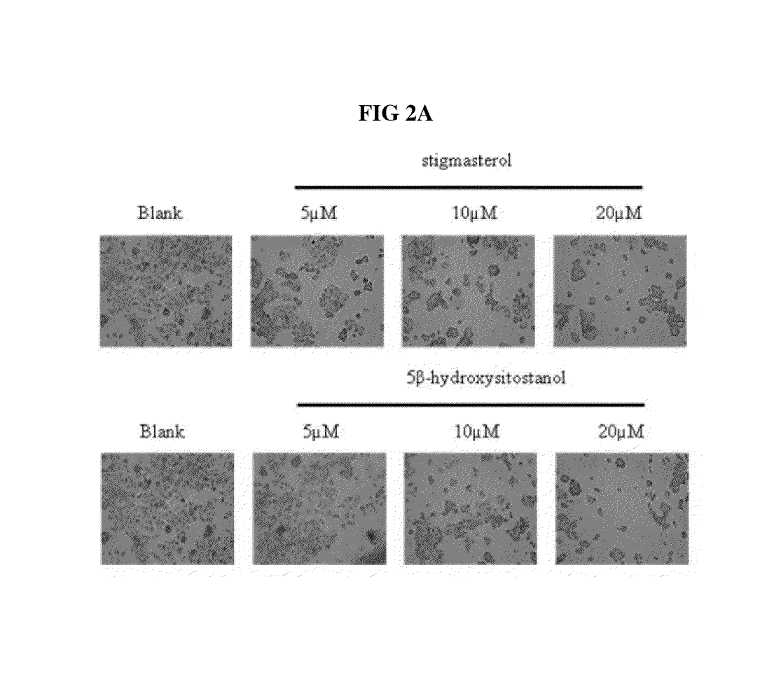

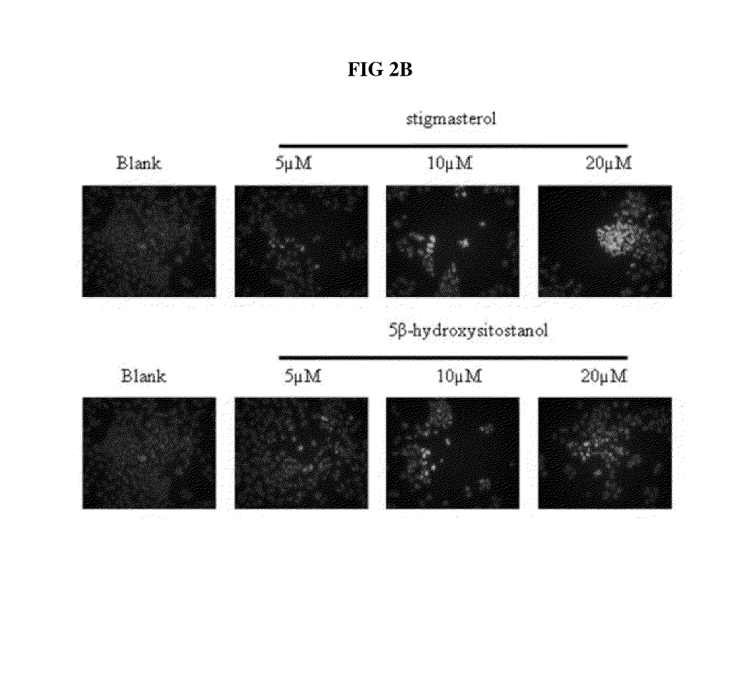

Referring to FIG. 2a, the number of attached cells (i.e., living cells) was remarkably reduced with increasing doses of stigmasterol and 5β-hydroxysitostanol. Treatment of stigmasterol and 5β-hydroxysitostanol also caused shrinkage of the attached HepG2 cell shape.

Further, the HepG2 cells were grown in 12-well plates at a density of 2×10⁴ cells/well. After overnight incubation, cells were treated with different concentrations (5 μM, 10 μM, 20 μM) of stigmasterol and 5β-hydroxysitostanol and incubated for another 24 h. The cells were then washed with PBS. After washing, 4% (v/v) formaldehyde solution was dissolved in PBS and added to the plate, and left for 1 h at room temperature so that cells were fixated. The fixed cells were stained with staining dye, Bisbenzimide Hoechst 33342 and left for 1 h at room temperature. The stained cells were observed under fluorescence microscope.

Referring to FIG. 2b, the nuclei of the attached HepG2 cells were significantly condensed according to stigmasterol and 5β-hydroxysitostanol with increasing concentrations. This observation corresponds to a typical apoptotic pathway.

### Experimental Example 3

### Cell Cycle Analysis

The HepG2 cells were cultured in 6 cm² dishes treated with different concentrations (5 μM, 10 μM, 20 μM) of stigmasterol and 5β-hydroxysitostanol. The cells were stained with propidium iodide (PI), and the cell cycle was analyzed by measuring the amount of DNA with FACS.

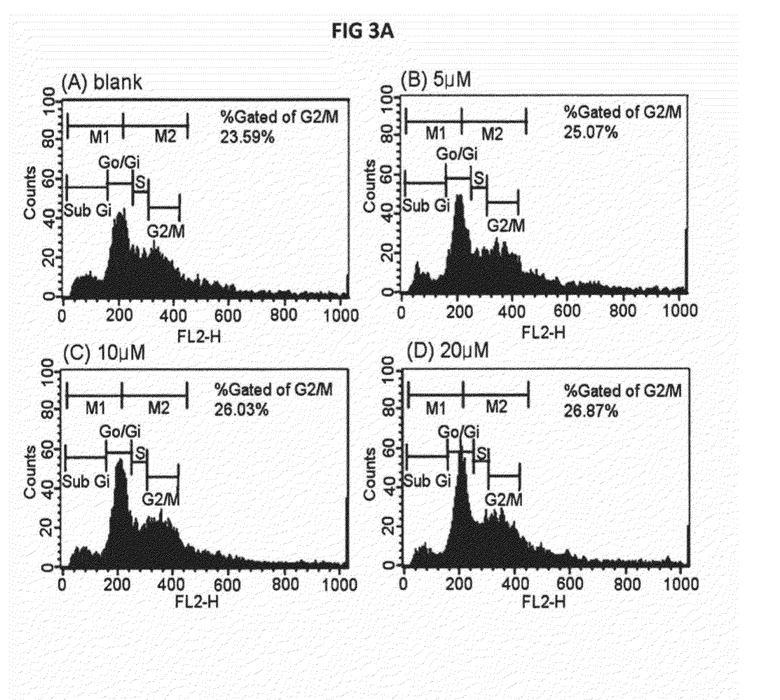

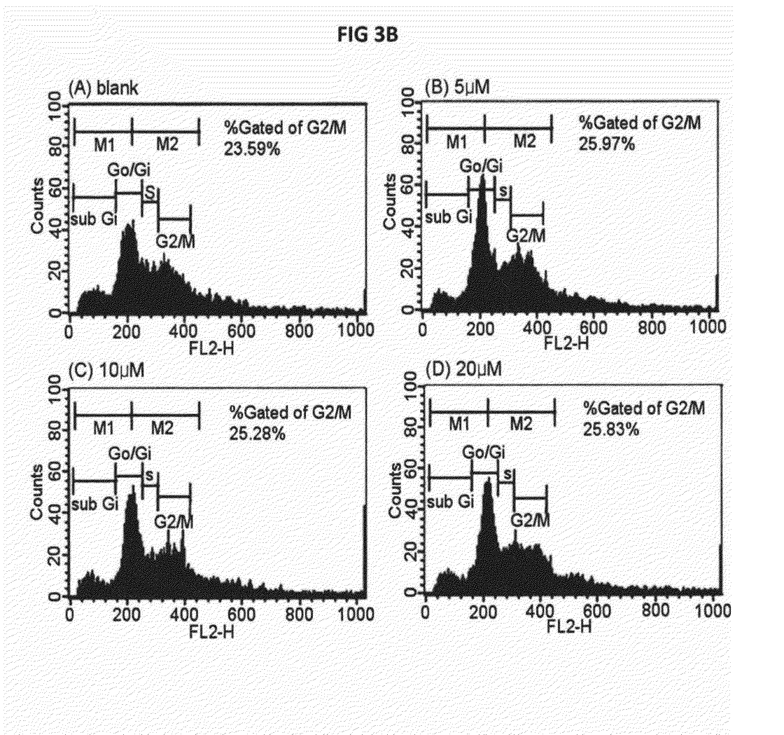

Referring to FIGS. 3a and 3b, we observed that the G2/M phase of the cell cycle increased as the concentration of stigmasterol and 5β-hydroxysitostanol increased. We could observe that G2/M phase was corresponding to 26.87% and 25.83% respectively at the highest concentration (20 μM). This confirms that stigmasterol and 5β-hydroxysitostanol inhibit circulation of cell dividing cycle to arrest HepG2 cells at G2/M phase thereby inhibiting proliferation of HepG2 cells.

### Experimental Example 4

### RT-PCR Analysis

HepG2 cells treated with stigmasterol and 5β-hydroxysitostanol were treated with TRIzol® reagent were transferred to microtubes at each concentration. 200 μl, of chloroform was added to each tube and microtubes were centrifuged at 12,000×g for 15 min. After centrifugation, supernatant phase was transferred to a new microtube and mixed with isopropanol at the ratio of 1:1. After incubation for 10 min at room temperature and centrifugation at 12,000×g for 10 min, supernatant was discarded and RNA pellet was washed, followed by centrifugation at 12,000×g for 15 min. Following removal of ethanol, RNA pellet was suspended in DEPC-treated water and so that RNA pellet was dissolved at 55° C. for 10 min. Dissolved RNA pellet was kept at −20° C. for further experiments.

RT-PCR was performed to check specific mRNA expression in HepG2 cells.

Thirteen μg of RNA dissolved in DEPC— treated water and 2 μL of oligo (di) were added to PCR microtubes and denaturated at 70° C. for 5 min in a thermal cycler. Later, RT-PCR mastermix was added to microtubes to synthesize cDNA. Primers corresponding to specific RNA were bound to the synthesized cDNA, and gene amplification was carried out with the thermal cycler. The PCR products were electrophoresed on 1% agarose gel and bands of the respective primers were checked with UV. Sequences of the RNA specific primers are shown in table 1.

TABLE 1

| primer | sequence | |
|---|---|---|
| Bax | Forward | 5′-TGC-CAG-CAA-ACT-GGT-GCT-CA-3′ |
| | Reverse | 5′-GCA-CTC-CCG-CCA-CAA-AGA-TG-3′ |
| Cas- | Forward | 5′-CAT-CCA-GTC-ACT-TTG-CCA-GA-3′ |
| pase-8 | Reverse | 5′-GCA-TCT-GTT-TCC-CCA-TGT-TT-3′ |
| Cas- | Forward | 5′-AAG-ACC-ATG-GCT-TTG-AGG-TG-3′ |
| pase-9 | Reverse | 5′-CAG-GAA-CCG-CTC-TTC-TTG-TC-3′ |
| p21 | Forward | 5′-CTG-TCA-CAG-GCG-GTT-ATG-AA-3′ |
| | Reverse | 5′-TGT-GCT-CAC-TTC-AGG-GTC-AC-3′ |
| p53 | Forward | 5′-GCG-CAC-AGA-GGA-AGA-GAA-TC-3′ |
| | Reverse | 5′-CTC-TCG-GAA-CAT-CTC-GAA-GC-3′ |
| FAS | Forward | 5′-TTG-CTG-GCA-CTA-CAG-AAT-GC-3′ |
| | Reverse | 5′-AAC-AGC-CTC-AGA-GCG-ACA-AT-3′ |
| FASL | Forward | 5′-CAC-TAC-CGC-TGC-CAC-CCC-3′ |
| | Reverse | 5′-CCA-GAG-AGA-GCT-CAG-ATA-CGT- |
| | | TG-3′ |
| XIAP | Forward | 5′-GAA-GAC-CCT-TGG-GAA-CAA-CA-3′ |
| | Reverse | 5′-CGC-CTT-AGC-TGC-TCT-TCA-GT-3′ |
| Bcl-2 | Forward | 5′-ATA-CCT-GGG-CCA-CAA-GTG-AG-3′ |
| | Reverse | 5′-TGA-TTT-GAC-CAT-TTG-CCT-GA-3′ |
| β-actin | Forward | 5′-GCC-ACC-CAG-AAG-ACT-GTG-GAT-3′ |
| | Reverse | 5′-TGG-TCC-AGG-GTT-TCT-TAC-TCC-30′ |

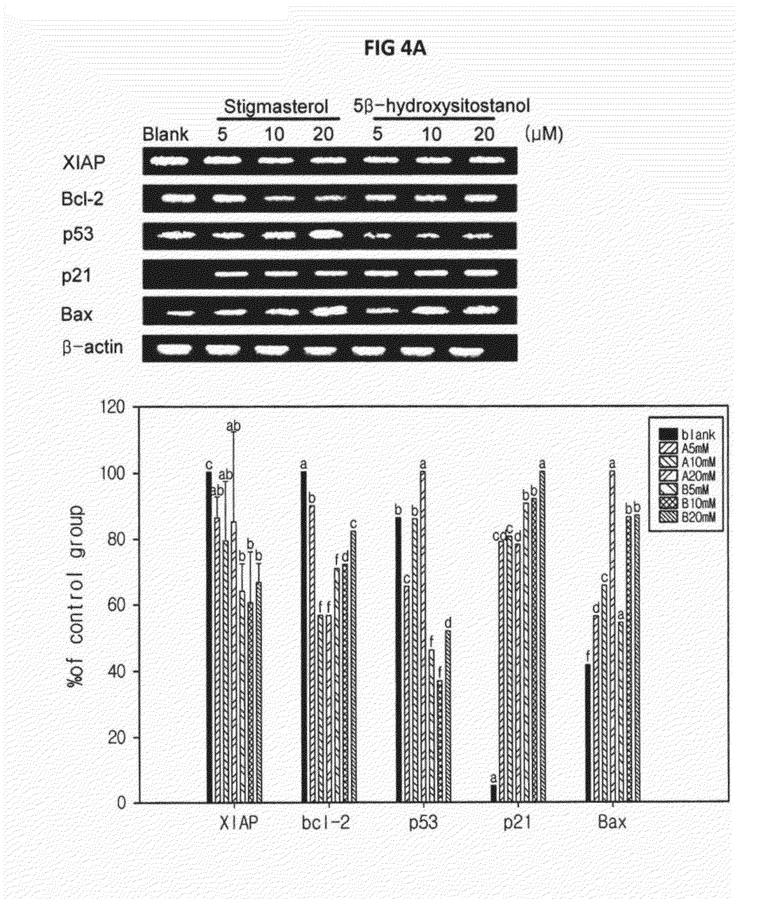

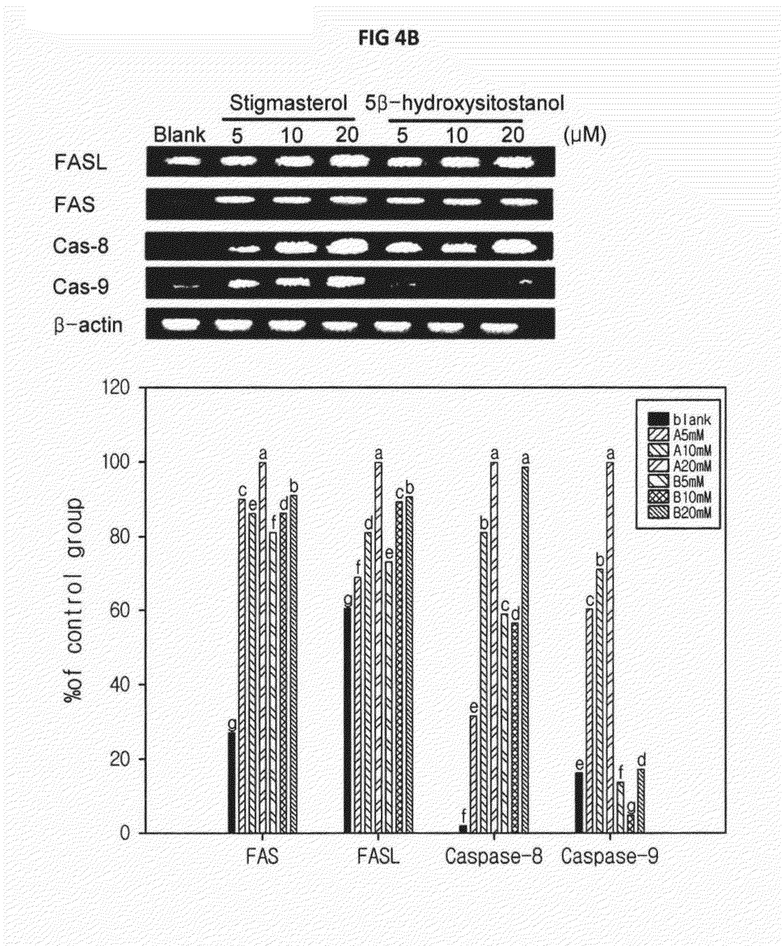

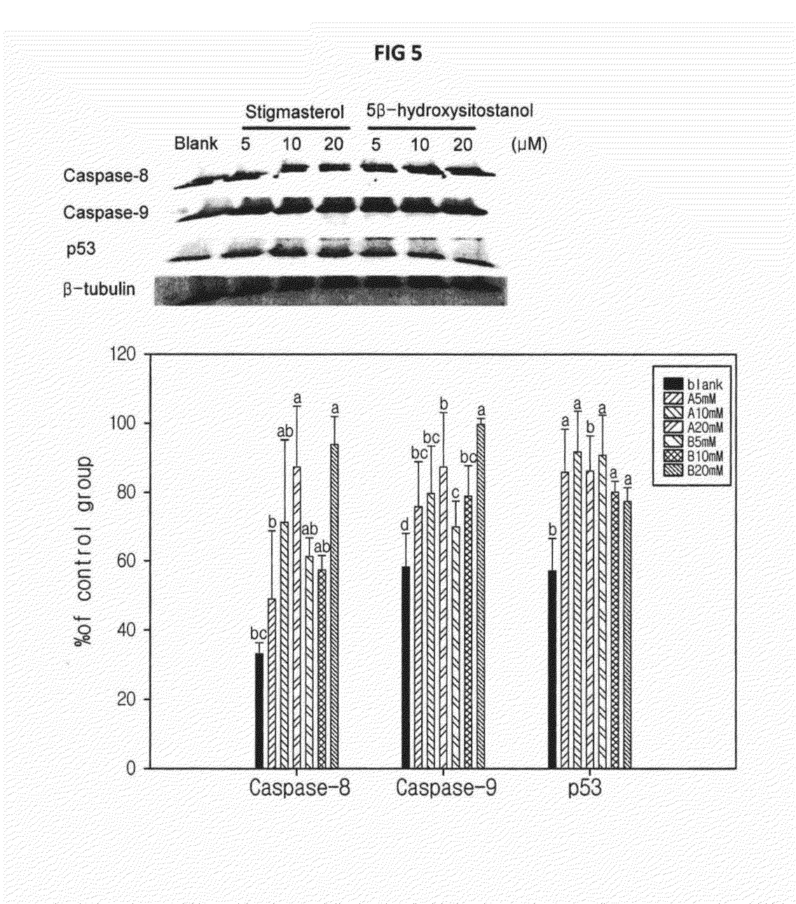

Referring to FIGS. 4 and 5, the inventors could confirm that stigmasterol upregulates expression of cell apoptosis inducing factors like caspase-8, 9, Bax, p53, p21, FAS and FASL, and downregulates the expression of cell apoptosis inhibiting factors which are Bcl-2 and XIAP.

It was also confirmed that 5β-hydroxysitostanol upregulates expression of caspase-8, Bax and FASL. According to these findings, the present inventors could confirm that stigmasterol upregulates the apoptosis inducing genes, downregulates apoptosis inhibiting factors, to thereby lead into apoptosis and death of hepatoma (HepG2) cells, and that 5β-hydroxysitostanol has no cytotoxicity according to concentration and upregulates the apoptosis inducing genes to thereby induce cell apopotosis.

### Experimental Example 5

### Western Blotting Assay

Western blotting assay was performed to investigate specific mRNA expression of HepG2 cells.

HepG2 cells were cultured at a density of 1×10⁴ cells/ml in 10 cm² culture dishes for 24 h. The cells were then treated with different concentrations (5 μM, 10 μM, 20 μM) of stigmasterol and 5β-hydroxysitostanol for 24 h. The cells were lysed in cell lysis buffer to give protein extract. The extracted protein was separated by molecular weight using SDS electrophoresis. Corresponding antibodies were attached to intended protein and the protein bands were detected with ECL solution using LAS-3000 system.

Referring to FIG. 6, it was confirmed that stigmasterol and 5β-hydroxysitostanol upregulate expression of cell apoptosis inducing factors, i.e., caspase-8, 9 genes.

While the present invention has been particularly shown and described with reference to exemplary embodiments thereof, it will be understood by those skilled in the art that various changes in form and details may be made therein without departing from the spirit and scope of the invention as defined by the appended claims.

1. A method of treating liver cancer comprising administering to a subject in need thereof a 5β-hydroxysitostanol as an active ingredient.

2. The method of claim 1, wherein the 5β-hydroxysitostanol is isolated from Navicula incerta.

3. The method of claim 1, wherein the 5β-hydroxysitostanol upregulates gene expression of one or more gene selected from the group consisting of caspase-8, 9, Bax, p53, p21, FAS and FASL of hepatoma cells.

4. The method of claim 1, wherein the 5β-hydroxysitostanol upregulates gene expression of one or more gene selected from the group consisting of Bcl-2 and XIAP of hepatoma cells.

5. A method of causing apoptosis in hepatoma cell comprising contacting the cell with 5β-hydroxysitostanol.

6. The method according to claim 5, wherein the cell is human cell.
